(19) United States
(12) Patent Application Publication
(10) Pub. No.: US 2019/0021884 A1
(43) Pub. Date: Jan. 24, 2019

(54) PROSTHETIC SOCKET RETENTION SYSTEMS AND METHODS

(71) Applicant: EPOCH MEDICAL INNOVATIONS, INC., Seattle, WA (US)

(72) Inventors: Michael Thomas BALLAS, Snohomish, WA (US); Gary Joseph BALLAS, Redmond, WA (US)

(21) Appl. No.: 15/993,272

(22) Filed: May 30, 2018

Related U.S. Application Data
(63) Continuation of application No. PCT/US2016/064464, filed on Dec. 1, 2016.
(60) Provisional application No. 62/261,735, filed on Dec. 1, 2015.

(51) Int. Cl. A61F 2/80; A61F 2/78

(52) U.S. Cl. CPC A61F 2/80; A61F 2002/802; A61F 2/7812

(57) ABSTRACT

A friction-based prosthetic retention system includes a prosthetic socket and a plurality of hydraulic actuators attached to an interior surface of the prosthetic socket. A socket liner is positioned over the plurality of hydraulic actuators and includes a plurality of friction elements. A limb liner is placed over a residual limb to be secured within the prosthetic socket. The limb liner includes a plurality of friction pad projections arranged to engage with the plurality of friction elements on the socket liner. A controller is attached to the prosthetic socket and is fluidly coupled to the plurality of hydraulic actuators. The controller is configured to control the plurality of hydraulic actuators to apply pressure to the socket liner to engage the plurality of friction elements of the socket liner with the friction pad projections of the limb liner to retain the prosthetic socket.

### PRIORITY DATA

The present application claims the benefit of US Provisional Patent Application No. 62/261,735, filed Dec. 1, 2015, which application is incorporated by reference herein in its entirety.

### RELATED APPLICATION DATA

The present application is related to U.S. patent application Ser. No. 14/855,248, filed Sep. 15, 2015, which application is incorporated by reference herein in its entirety.

### TECHNICAL FIELD

The present disclosure relates generally to prosthetic devices, and more particularly to retention systems and methods for prosthetic devices.

### BACKGROUND ART

Current prosthetic retention systems for securing a prosthetic socket to a partial limb typically include a lock pin that is part of a limb liner. A user places the limb liner over his or her residual limb and then inserts the lock pin on the limb liner into a corresponding mating portion of the prosthetic socket to thereby secure the residual limb to the socket and align the residual limb within the socket. This lock pin type approach may make it difficult for the user to properly align the lock pin with the mating portion of the prosthetic socket. Any such misalignment may result in the residual limb not being properly aligned within the socket, which may cause pressure spots that result in discomfort for the user. As a result, improved prosthetic socket retention systems are needed.

### BRIEF SUMMARY

One embodiment of the present disclosure is directed to a friction-based retention system including a prosthetic socket and a plurality of hydraulic actuators attached to an interior surface of the prosthetic socket. A socket liner is positioned over the plurality of hydraulic actuators and includes a plurality of friction elements. A limb liner is placed over a residual limb to be secured within the prosthetic socket. The limb liner includes a plurality of friction pad projections arranged to engage with the plurality of friction elements on the socket liner. A controller is attached to the prosthetic socket and is fluidly coupled to the plurality of hydraulic actuators. The controller is configured to control the plurality of hydraulic actuators to apply pressure to the socket liner to engage the plurality of friction elements of the socket liner with the friction pad projections of the limb liner to retain the prosthetic socket.

### BRIEF DESCRIPTION OF THE SEVERAL VIEWS OF THE DRAWINGS

FIG. 1 is a cross-sectional view of a prosthetic device including a friction-based prosthetic retention system according to one embodiment of the present invention.

FIG. 2 is a more detailed cross-sectional view of the friction-based prosthetic retention system of FIG. 1 according to an embodiment of the present disclosure.

FIG. 3A is a more detailed cross-sectional view of the friction-based prosthetic retention system of FIG. 2 in which the limb liner further includes an elastomeric end stop according yet another embodiment of the present disclosure.

FIG. 3B is vertical cross-sectional view of the friction-based prosthetic retention system of FIG. 3A illustrating a circumferential lever-lock ring of the prosthetic socket according to one embodiment of the present disclosure.

FIG. 3C is perspective view of the elastomeric end stop of FIG. 3A showing a circumferential groove in which fits the circumferential lever-lock ring of FIG. 3B according to one embodiment of the present disclosure.

FIG. 4A illustrates a portion of the socket liner of FIGS. 2 and 3A-3B and shows in more detail an embodiment of the interlocking friction mechanism formed by an arrangement of friction pad projections on the limb liner and a wavy configuration of ridges and valleys on the socket liner.

FIG. 4B is a side view of a portion of the socket liner and limb liner of FIG. 4A illustrating the frictional forces developed due to the interaction or meshing of the friction pad projections 114 on the limb liner and the ridges 400 and valleys 402 on the socket liner.

FIG. 5 illustrates a portion of a limb liner according to another embodiment of the present disclosure in which the limb liner includes multiple friction pads each including arrays of friction pad projections.

FIG. 6A illustrates a conventional prosthetic socket and lock pin arrangement with the residual limb properly positioned within the prosthetic socket.

FIG. 6B illustrates misalignment of the residual limb within the prosthetic socket that commonly occurs in the conventional lock pin arrangement.

### DETAILED DESCRIPTION

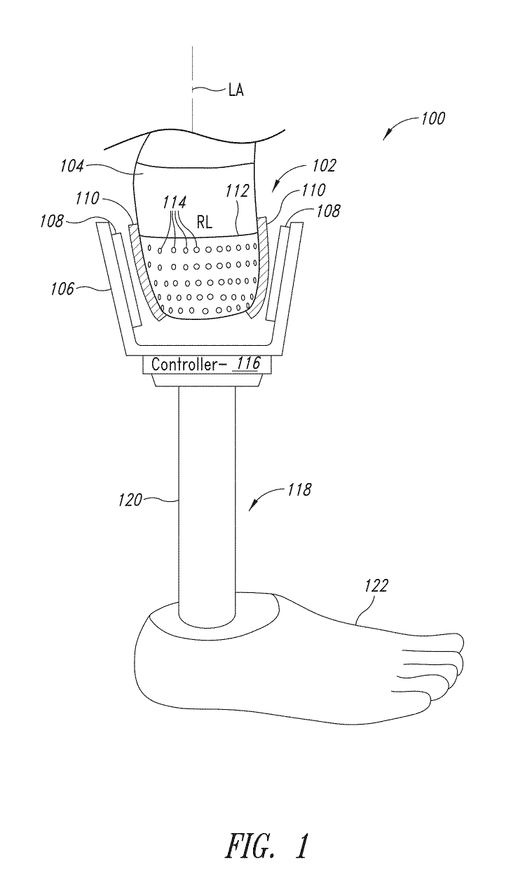

FIG. 1 is a cross-sectional view of a prosthetic device 100 including a friction-based prosthetic retention system 102 according to one embodiment of the present invention. An amputee or user (these terms will be used interchangeably herein) utilizes the prosthetic device 100 to replace a missing body part, which in the example of FIG. 1 corresponds to the lower portion of a leg of the user. One skilled in the art will realize that although the embodiment of FIG. 1 and the other embodiments described in the following description are for a missing portion of a leg of a user, in other embodiments the prosthetic device 100 replaces other parts of the user's body, such as a foot, a hand, or an arm. In operation or use, a user places his or her residual limb RL inside a limb liner 104 that covers the end or distal portion of the residual limb that is then inserted into a prosthetic socket 106. More specifically, the inside of the prosthetic socket 106 includes fluid bladders 108 attached to the interior surface of the prosthetic socket and a socket liner 110 positioned within the socket between the fluid bladders and the limb liner 104. The limb liner 104, fluid bladders 108 and socket liner 110 together form the friction-based prosthetic retention system 102 that retains the socket 106 on the residual limb RL, as will be described in more detail below.

In the present description, certain details are set forth in conjunction with the described embodiments to provide a sufficient understanding of the present disclosure. One skilled in the art will appreciate, however, that the subject matter of the present disclosure may be practiced without these particular details. Furthermore, one skilled in the art will appreciate that the example embodiments described below do not limit the scope of the present disclosure to the disclosed embodiments, and will also understand that various modifications, equivalents, and combinations of the disclosed embodiments and components of such embodiments are within the scope of the present disclosure. Embodiments including fewer than all the components of any of the respective described embodiments may also be within the scope of the present disclosure although not expressly described in detail below. Finally, the operation of well-known components and/or processes have not been shown or described in detail below to avoid unnecessarily obscuring the present disclosure.

The limb liner 104 is made from suitable material to provide some protection for the distal portion of the residual limb RL to thereby make the prosthetic socket more comfortable for the user to wear on the residual limb. The prosthetic socket 106 is made from a suitably rigid material. The limb liner 104 also includes a friction pad 112 positioned on the end portion of the limb liner that covers the distal portion of the residual limb RL as shown in the figure. This friction pad 112 includes a plurality of friction pad projections 114 arranged on the friction pad and which function in generating a friction force that retains the residual limb RL in the socket 106, as will be described in more detail below.

The socket liner 110 is formed from a suitable flexible material and is positioned between the fluid bladders 108 and limb liner 104. The fluid bladders 108 are hydraulic actuators and are operable to expand and contract in response to fluid being pumped into or removed from the bladders to thereby control the pressure that is applied to the residual limb RL to hold the residual limb in place (i.e., retain the residual limb) within the socket 106. A controller 116 is fluidly coupled to the fluid bladders 108 to control the expansion and contraction of these hydraulic actuators. This coupling between the controller 116 and the fluid bladders 108 is not expressly illustrated in FIG. 1 to simplify the figure. The controller 108 is physically attached to the bottom of the socket 106 in the embodiment of FIG. 1 but may be positioned in different locations in other embodiments, and need not even be attached to the socket so long as the controller is properly coupled to control the hydraulic actuators or fluid bladders 108. The prosthetic device 100 further includes a limb portion 118 that includes a post 120 and an artificial foot 122 attached at an end of the post in the embodiment of FIG. 1. The specific structure of the limb portion 118 may of course vary in different embodiments of the present disclosure. The post 120 has a longitudinal axis that extends approximately parallel to a longitudinal axis LA of the residual limb RL as illustrated in FIG. 1.

The prosthetic device 100 may be retrofitted to existing prosthetic devices including socket and limb portions. The socket 106 is custom-designed for each user so that the socket properly fits onto the distal end of the residual limb RL with the limb liner 104 placed over the residual limb. A prosthetist custom designs the socket 106 for each user. Where the prosthetic device 100 is retrofitted into an existing socket the prosthetist would arrange and attach the hydraulic actuators or fluid bladders 108 and the socket liner 110 on the interior of the socket, and would also interconnect the fluid bladders to the controller 116. The prosthetist would also attach the controller 116 to the socket 106 and attach the post 120 to the controller as shown in the figures. This could be done in the form of a kit including the fluid bladders 108 and controller 116 along with any other required components for allowing the prosthetist to more easily retrofit an existing socket. In other embodiments, the hydraulic actuators or fluid bladders 108 and controller 116 are formed as part of the socket 116 when the custom-designed socket is being made the user. In this situation, the fluid bladders 108 and controller 116 may be an integral part of the socket 106. For example, electronic components of the controller 108 could be integrally formed in the socket 104 as could the required hydraulic lines interconnecting the hydraulic actuators 106 and the controller, and the same is true of the hydraulic actuators themselves.

The hydraulic actuators or fluid bladders 108 may have different structures and arrangements within the socket 106 in different embodiments. In other embodiments, different types of actuators, such as pneumatic actuators, are utilized instead of hydraulic actuators and the controller 116 operates to control these actuators to secure the residual limb RL in the socket 106. The controller 116 may sense a variety of different parameters, such as pressure applied to the residual limb, ambulatory state of the user, and so on, in controlling the fluid bladders 108 or other types of actuators. The controller 116 includes all sensors and any other components required to implement the specific control approach being utilized to control the pressure applied to the residual limb RL through expansion and contraction of the fluid bladders 108. The controller 116 may be formed from a variety of different types of components, such as electronic circuitry including hardware, software, firmware, and a combination of all of these, in addition to other components such as pumps and sensors.

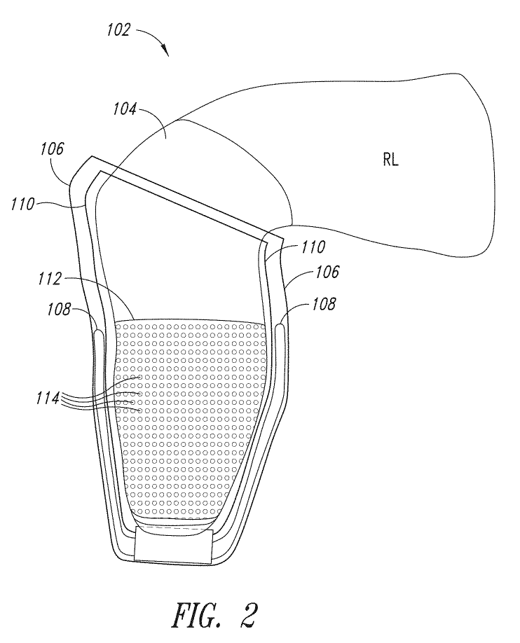

FIG. 2 is a more detailed cross-sectional view of the friction-based prosthetic retention system 102 of FIG. 1 formed by the limb liner 104, fluid bladders 108 and socket liner 110. Instead of using a lock pin to ensure a residual limb RL is retained as in conventional prosthetic devices, the friction-based prosthetic retention system 102 utilizes an interlocking friction mechanism formed by the friction pad projections 114 on the limb liner 104 and corresponding friction elements (not shown in FIG. 2) on the socket liner 110 in securing the residual limb RL in the prosthetic socket 106. The friction pad projections 114 on the friction pad 112 are configured to interface with the friction elements (not shown) on the socket liner 110 as the fluid bladders 108 expand and apply pressure to the socket liner to thereby secure the residual limb RL within the prosthetic socket 106.

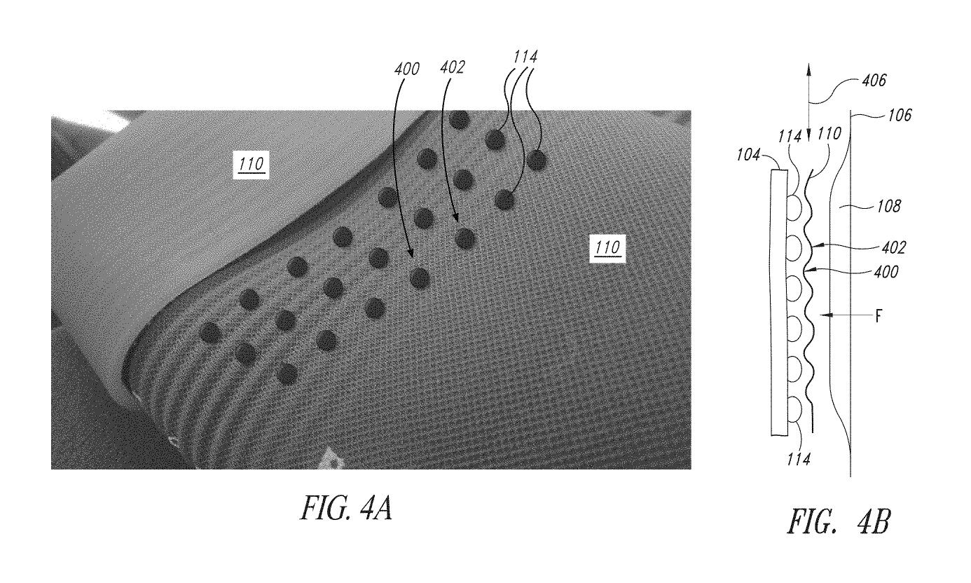

An adaptive compression system as described in U.S. patent application Ser. No. 14/855,248 may be implemented by the controller 116 in controlling the friction-based prosthetic retention system 102. The controller 116 controls the fluid bladders 108 in the prosthetic socket 106 to either pump fluid into the bladders or remove fluid from the bladders to thereby apply desired pressure through the socket liner 110 and limb liner 104. In this way, the controller 116 secures the residual limb RL within the prosthetic socket 106 and allows a user to release the residual limb RL from the prosthetic socket 106 when the user desires to do so, such as when the user is sleeping. The pressure applied through expansion of the fluid bladders 108 results in sufficient force between the friction pad projections 114 on the friction pad 112 of the limb liner 104 and the friction elements (not shown) on the socket liner 110 to securely hold the residual limb in place within the prosthetic socket. Different patterns for the friction pad projections 114 may of course be utilized, and the friction pads projections themselves may have shapes other than the rounded shape illustrated in the figure, such as cylindrical-shaped projections, spherical-shaped projections, pointed projections, and so on. The same is true of the corresponding friction elements on the socket liner 110 as will be described in more detail below with reference to FIGS. 4A and 4B.

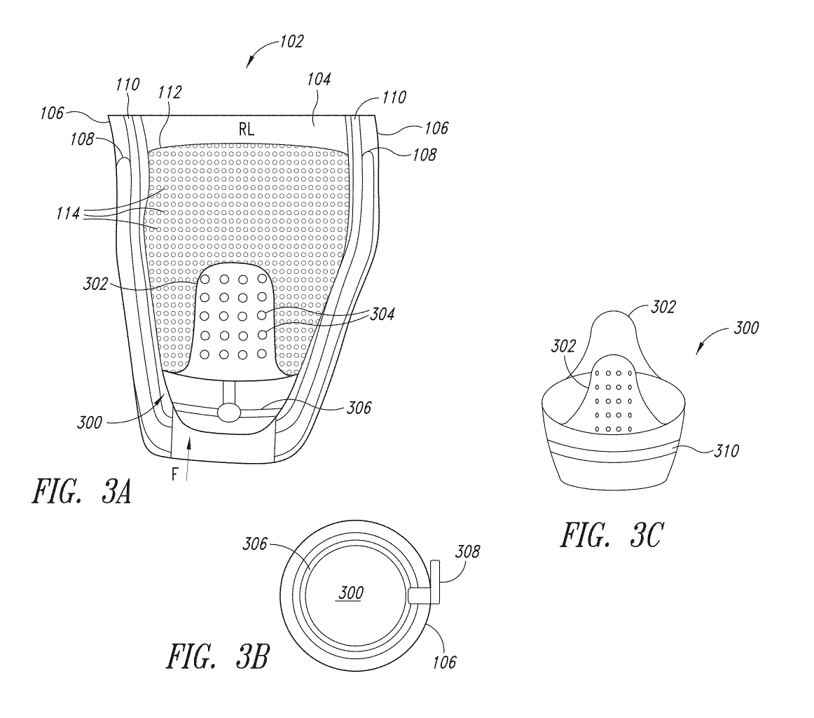

FIG. 3A is a more detailed cross-sectional view of the friction-based prosthetic retention system 102 of FIG. 2 in which the limb liner 104 further includes an elastomeric end stop 300 according yet another embodiment of the present disclosure. The elastomeric end stop 300 is made from a suitable elastomeric or other suitable material and functions provide some additional cushioning from vertical forces F against the distal end of the residual limb RL, and in this way, improves the comfort of the user. In addition, the elastomeric end stop 300 is circular-shaped and includes a circumferential groove (not shown in FIG. 3A) that operates in combination with a circumferential lever-lock ring 302 attached to the prosthetic socket 106 to help properly center the residual limb RL within the prosthetic socket, as will be described in more detail below with reference to FIGS. 3B and 3C. The elastomeric end stop 300 includes upper tabs 302, one of which is shown in FIG. 3A, that extend along the longitudinal axis LA of the residual limb RL inserted into the limb liner 104 on which the elastomeric end stop 300 formed. Each upper tab 302 of the elastomeric end stop 300 includes a plurality of grip projections 304 that function to provide additional frictional force between the elastomeric end stop and the socket liner 110 to help retain the residual limb RL in the prosthetic socket 106.

FIG. 3B is vertical cross-sectional view of the friction-based prosthetic retention system 102 of FIG. 3A illustrating a circumferential lever-lock ring 306 contained in the prosthetic socket 106 according to one embodiment of the present disclosure. FIG. 3B thus shows a view looking downward into the prosthetic socket 106 of FIG. 3A. The circumferential lever-lock ring 306 is positioned at the bottom of the prosthetic socket 106 and attached to the socket, and includes a lever-lock handle 308 that extends through the prosthetic socket 106 from the interior surface to the exterior surface as seen in the figure. Thus, the lever-lock handle 308 and is exposed on the exterior surface of the prosthetic socket 106. FIG. 3C is perspective view of the elastomeric end stop 300 of FIG. 3A showing a circumferential groove 310 that is configured to receive the circumferential lever-lock ring 306 of FIG. 3B. The elastomeric end stop 300 includes two upper tabs 302 in the embodiment of FIG. 3C but could include additional upper tabs of varied shape in additional embodiments.

In operation, a user inserts his or her residual limb RL with the limb liner 104 placed over the distal end of that residual limb into the prosthetic socket 106. As the residual limb RL is placed down into the prosthetic socket 106 the tapered circular shaped bottom of the elastomeric end stop 300 goes through the circumferential lever-lock ring 306 positioned in centered in the distal portion of the prosthetic socket. In this way, as the distal end or bottom of the elastomeric end stop 300 goes through the circumferential lever-lock ring 306 the residual limb RL is centered within the prosthetic socket 106. The user pushes the residual limb RL down into the prosthetic socket 106 until the user feels the circumferential lever-lock ring 306 engage with the circumferential groove 310 formed in the elastomeric end stop 300. At this point, the user then actuates the lever-lock handle 308, causing the circumference of the circumferential lever-lock ring 306 to reduce such that the ring tightens around the end stop 300 and is forced into the circumferential groove 310 of the end stop. In this way, the residual limb RL is properly positioned, centered in the prosthetic socket 106. The engagement of the circumferential lever-lock ring 306 with the circumferential groove 310 also helps retain the residual limb RL within the prosthetic socket 106. One skilled in the art will understand suitable materials for forming the circumferential lever-lock ring 306 and lever-lock handle 308. The controller 116 thereafter controls the fluid bladders 108 to apply pressure to engage the friction pad projections 114 on the limb liner 104 and the friction elements on the socket liner 110 to retain the residual limb RL within the prosthetic socket 106.

FIG. 4A illustrates a portion of the socket liner 110 of FIGS. 2 and 3A-3C and shows in more detail an embodiment of the interlocking friction mechanism formed by the friction pad projections 114 on the limb liner 104 and the friction elements on the socket liner 110, which are formed by a wavy configuration of ridges 400 and valleys 402 on the socket liner 110 in the embodiment of FIG. 4A. FIG. 4B is a side view of a portion of the socket liner 110 and limb liner 104 of FIG. 4A illustrating the frictional force developed due to the interaction or meshing of the friction pad projections 114 on the limb liner and the ridges 400 and valleys 402 on the socket liner. An arrow 406 indicates the vertical direction or direction along the longitudinal axis LA of the residual limb RL in FIG. 4B. In operation, as the fluid bladders 108 are expanded a force F and pressure is exerted by the fluid bladders against the socket liner 110. This force F applied to the socket liner 110 pushes the socket liner against the friction pad projections 114 of the limb liner 104. As a result of this force and slight movement of the socket liner 104 in the vertical direction 406 relative to the socket liner 110, the friction pad projections 114 of the limb liner 104 are pushed into the valleys 402 in the socket liner 110 and the ridges 400 in the socket liner are between adjacent friction pad projections. In this way, the friction elements of the socket liner 104 engage or mesh with the friction pad projections 114 on the limb liner 104 and the frictional force between the two functions to retain the residual limb RL in the prosthetic socket 106. The friction pad projections 114 are accordingly arranged on the limb liner 104 in a pattern to properly engage with the pattern of the friction elements on the socket liner 110. Different patterns for both the friction pad projections 114 and friction elements on the socket liner 110 may of course be utilized, so long as the each of the patterns is configured to engage or mesh with the other of the patterns. Both the limb liner 104 and socket liner 110 include friction pad projections 110 in other embodiments of the present disclosure.

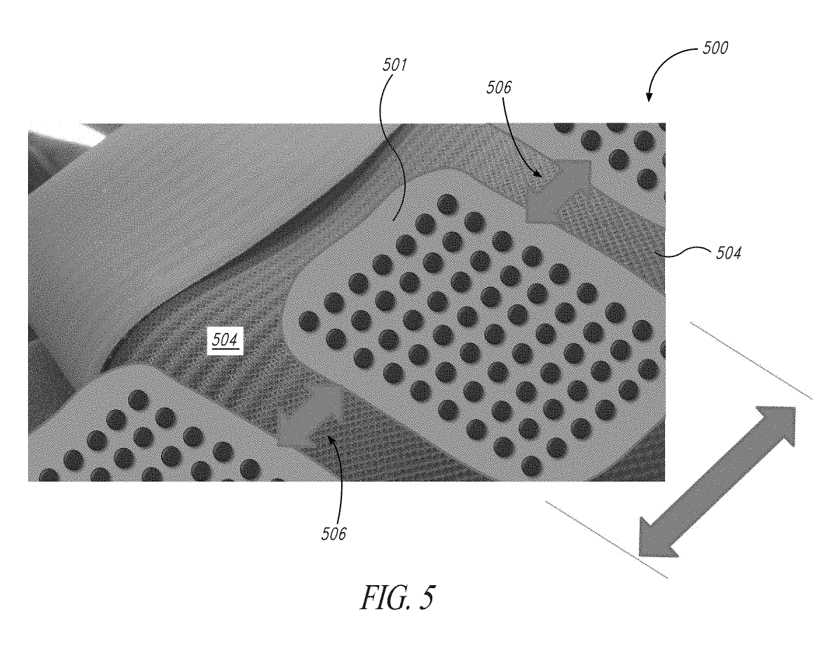

FIG. 5 illustrates a portion a limb liner 500 according to another embodiment of the present disclosure in which the limb liner includes multiple friction pads 500, each including arrays of friction pad projections 502. The friction pads 500 are arranged spaced apart on an elastic material 504 forming the limb liner 104, with stretch zones 506 of the elastic material being defined between adjacent friction pads 500. The limb liner 500 is thus an alternative to the embodiment of the limb liner 104 of FIGS. 1,2 and 3A in which the limb liner includes friction projection pads 114 over substantially the entire surface of the limb liner. The friction pads 500 could be formed as an integral part of the limb liner 500 or could be attached to the elastic material 504 where the elastic material is a conventional limb liner.

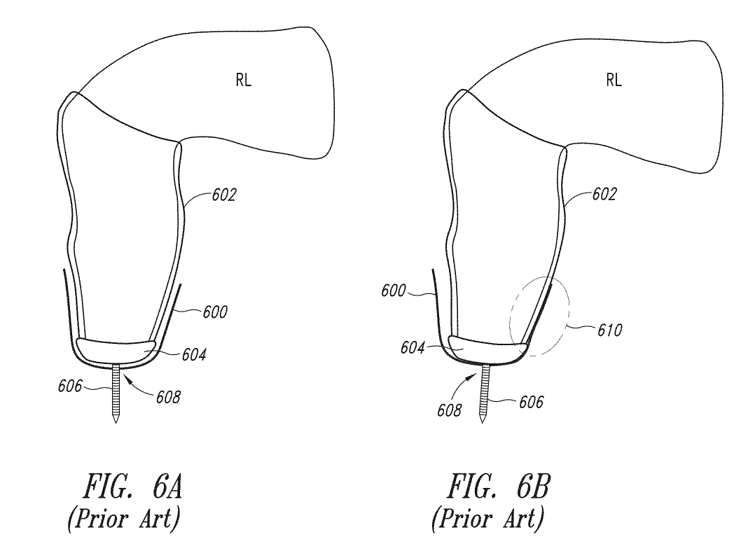

FIG. 6A illustrates a conventional prosthetic socket 600 and lock pin arrangement a residual limb RL properly positioned within the prosthetic socket. A limb liner 602 is place over the distal end of the residual limb RL and has a cushioning end portion 604 to which a lock pin 606 is attached. The limb portion of the prosthesis, which attaches to the lock pin 606, is not shown in FIG. 6A. To attach the prosthetic socket 600 to the residual limb RL, the user inserts the lock pin 606 through a hole 608 the bottom portion of the prosthetic socket 600. FIG. 6A shows the residual limb RL properly positioned within the prosthetic socket 600, with the lock pin 606 extending vertically or longitudinally through the hole 608 and the residual limb being centered within the prosthetic socket.

In contrast, FIG. 6B illustrates misalignment of the residual limb RL within the prosthetic socket 600 that commonly occurs with the conventional lock pin arrangement. When the user in attempting to insert the lock pin 606 through the hole 608, the pin can extend through hole in a misaligned way that results in the residual limb RL being misaligned and not centered within the prosthetic socket 600. This misalignment results in high-pressure spots 610 on certain portion of the residual limb RL, as illustrated in FIG. 6B. These high-pressure spots 610 can result in discomfort for the user and, even where the user experiences no discomfort, are not good for the health of the residual limb RL. The a friction-based prosthetic retention system disclosed in the present disclosure reduces the occurrence of such high pressure spots, and also eliminates the need for a lock pin to reliably secure the prosthetic device to the residual limb of a user.

The friction-based prosthetic retention system 102 of the present disclosure address several fundamental problems existing users typically experience, such as “pistoning” of the residual limb RL within the prosthetic socket 106 where the residual limb undesirably moves up and down within the prosthetic socket. Another problem users experience is centering of the distal end of the residual limb RL relative to the prosthetic socket 106. In existing systems, poor lock pin alignment can create pressure hotspots as discussed above with reference to FIGS. 6A and 6B and make it difficult to actually engage the lock pin receptacle in the prosthetic socket.

The friction-based prosthetic retention system 102 reduces slip of the residual limb RL relative to the prosthetic socket 106 and includes an elastomeric end stop 300 that employs a circumferential lever-lock ring 306 to center the residual limb RL and to provide definitive locking of limb relative to prosthetic socket 106. In one embodiment, the friction pad system of the limb liner 104 and the elastomeric end stop 300 are a monolithic element, as shown in the embodiment of FIG. 3A. The general shape of the disclosed embodiments allows the limb liner 104 to self-center on the residual limb RL.

The friction-based prosthetic retention system 102 works in conjunction with the powered adaptive socket interface system including the fluid bladders 108 since this powered adaptive socket interface system has the ability, via deflation of the bladders 108, to release from the friction pad projections 114 on the limb liner 104.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A prosthetic system, comprising:
a prosthetic socket having an interior surface and an exterior surface;
a plurality of hydraulic actuators attached to the interior surface of the prosthetic socket;
a socket liner positioned over the plurality of hydraulic actuators, the socket liner including a plurality of friction elements;
a limb liner configured to be placed over a residual limb to be secured within the prosthetic socket, the limb liner including a plurality of friction pad projections arranged to engage with the plurality of friction elements on the socket liner; and
a controller attached to the prosthetic socket and fluidly coupled to the plurality of hydraulic actuators, the controller configured to control the plurality of hydraulic actuators to apply pressure to the socket liner to engage the plurality of friction elements of the socket liner with the friction pad projections of the limb liner to retain the prosthetic socket on the residual limb in the limb liner.

2. The prosthetic system of claim 1, wherein the limb liner further comprises an elastomeric end stop formed on a distal end of the limb liner, the distal end of the limb liner configured to be positioned over the distal end of the residual limb when the limb liner is in position on the residual limb.

3. The prosthetic system of claim 2, wherein the elastomeric end stop is circular-shaped and further comprises a circumferential groove, and wherein the prosthetic socket further comprises a circumferential lever-lock ring configured to engage with the circumferential groove.

4. The prosthetic system of claim 3, wherein the elastomeric end stop further comprises a plurality of upper tabs, each of the upper tabs including grip projections arranged to engage with the limb liner to assist in retaining the residual limb within the prosthetic socket in response to pressure applied by the hydraulic actuators.

5. The prosthetic system of claim 3, wherein the plurality of friction elements on the socket liner comprises a wavy configuration of ridges and valleys on the socket liner, the ridges being arranged to be positioned between adjacent friction pad projections on the limb liner and the valleys configured to receive the friction pad projections.

6. The prosthetic system of claim 1, wherein the plurality of hydraulic actuators comprises a plurality of fluid bladders.

7. The prosthetic system of claim 1 further comprising a limb portion attached to the prosthetic socket.

8. The prosthetic system of claim 7, wherein the limb portion comprises a prosthetic foot.

9. The prosthetic system of claim 1, wherein the controller comprises electronic circuitry to control the hydraulic actuators.

10. The prosthetic system of claim 1, wherein the plurality of friction pad projections on the limb liner and the plurality of friction elements on the socket liner each comprise a plurality of projections arrange in rows and columns.
